(19) United States
(12) Patent Application Publication
(10) Pub. No.: US 2019/0262001 A1
(43) Pub. Date: Aug. 29, 2019

(54) OCCLUSIVE MEDICAL DEVICE WITH SPLIT COLLAR

(71) Applicant: BOSTON SCIENTIFIC SCIMED, INC., Maple Grove, MN (US)

(72) Inventors: Thomas P. Jancaric, Maple Grove, MN (US); Hoi Ki Ricky Chow, New Brighton, MN (US); Kelsey Rae Cooper, Blaine, MN (US); Eric Dinges, Minneapolis, MN (US); John D. Kroeger, Mounds View, MN (US); Reggie Roth, Monticello, MN (US); Nicholas Lee Tassoni, Andover, MN (US); Marla Kae Waters, Ramsey, MN (US)

(73) Assignee: BOSTON SCIENTIFIC SCIMED, INC., MAPLE GROVE, MN (US)

(21) Appl. No.: 16/280,536

(22) Filed: Feb. 20, 2019

Related U.S. Application Data
(60) Provisional application No. 62/634,509, filed on Feb. 23, 2018.

(51) Int. Cl. A61B 17/12; A61B 90/00

(52) U.S. Cl. CPC A61B 17/12113; A61B 17/1214; A61B 2017/00526; A61B 17/12168; A61B 90/39

(57) ABSTRACT

An occlusive medical device such as a vascular implant includes a terminal portion including a first plurality of strut segments that are each joined together at a distal end thereof to form a closed end of the frame, an intermediate portion including a second plurality of strut segments, the second plurality of strut segments including a greater number of strut segments than the first plurality of strut segments, and a main portion including a third plurality of strut segments, the third plurality of strut segments including a greater number of strut segments than the second plurality of strut segments. The occlusive medical device may have an expanded configuration in which the second plurality of strut segments and the third plurality of strut segments together form a plurality of cells.

### CROSS-REFERENCE TO RELATED APPLICATIONS

This application claims the benefit of priority under 35 U.S.C. § 119 to U.S. Provisional Application Ser. No. 62/634,509, filed Feb. 23, 2018, the entirety of which is incorporated herein by reference.

### TECHNICAL FIELD

The present disclosure pertains generally to vascular occlusion devices and more particularly to vascular occlusion devices that are cut from a tubular member.

### BACKGROUND

Medical devices typically used for cardiovascular system treatments may involve complex and invasive therapies resulting in significant discomfort, pain, and long recovery times for patients. Recently, less invasive, percutaneous treatments have been developed. There is an ongoing need for improved, less invasive cardiovascular treatments.

### SUMMARY

The disclosure provides design, material, and manufacturing method alternatives for occlusive medical devices, particularly occlusive medical devices that may be used for occluding blood flow through a vessel. For example, the disclosure is directed to an occlusive medical device that includes a terminal portion including a first plurality of strut segments that are each joined together at an end thereof to form a closed end of the frame, an intermediate portion including a second plurality of strut segments, the second plurality of strut segments including a greater number of strut segments than the first plurality of strut segments and a main portion including a third plurality of strut segments, the third plurality of strut segments including a greater number of strut segments than the second plurality of strut segments. The occlusive medical device has an expanded configuration in which the second plurality of strut segments and the third plurality of strut segments together form a plurality of cells.

Alternatively or additionally, each strut segment of the first plurality of strut segments in the terminal portion may bifurcate into a pair of strut segments of the second plurality of strut segments in the intermediate portion.

Alternatively or additionally, each strut segment of the second plurality of strut segments in the intermediate portion may bifurcate into a pair of strut segments of the third plurality of strut segments in the main portion.

Alternatively or additionally, each of the first plurality of strut segments may be configured to come together to form part of a split collar.

Alternatively or additionally, each of the first plurality of strut segments may include an arcuate end portion, and the arcuate end portion of each of the first plurality of strut segments may combine to form the split collar.

Alternatively or additionally, the arcuate end portion of each of the first plurality of strut segments may be joined together to form an end of the occlusive medical device.

Alternatively or additionally, the occlusive medical device may further include a closed second end proximal of the main portion of the occlusive medical device.

Alternatively or additionally, the occlusive medical device may further include a membrane.

Alternatively or additionally, the occlusive medical device may default to the expanded configuration when unconstrained.

Another example of the disclosure is an occlusive medical device that includes a membrane and a support frame that supports the membrane. The support frame includes a terminal portion including a first plurality of strut segments that are each joined together at an end thereof to form a closed end of the frame, each of the first plurality of strut segments having a first width, the first width measured orthogonally to a length of each of the first strut segments, an intermediate portion including a second plurality of strut segments, each of the second plurality of strut segments having a second width that is less than the first width, the second width measured orthogonally to a length of each of the second strut segments and a main portion including a third plurality of strut segments, each of the third plurality of strut segments having a third width that is less than the second width, the third width measured orthogonally to a length of each of the third strut segments.

Alternatively or additionally, the second width may be less than half of the first width.

Alternatively or additionally, the third width may be about half of the second width.

Alternatively or additionally, each strut segment of the first plurality of strut segments in the terminal portion may bifurcate into a pair of strut segments of the second plurality of strut segments in the intermediate portion.

Alternatively or additionally, each strut segment of the second plurality of strut segments in the intermediate portion may bifurcate into a pair of strut segments of the third plurality of strut segments in the main portion.

Alternatively or additionally, each of the first plurality of strut segments may be configured to come together to form part of a split collar.

Alternatively or additionally, each of the first plurality of strut segments may form an arcuate end portion, and the arcuate end portion of each of the first plurality of strut segments may combine to form the split collar.

Another example of the disclosure is a method of manufacturing an occlusive medical device that includes laser cutting a tubular member to define a support frame. The support frame includes a terminal portion including a first plurality of strut segments, an intermediate portion including a second plurality of strut segments, where each of the strut segments of the first plurality of strut segments bifurcate to form two strut segments of the second plurality of strut segments, and a main portion including a third plurality of strut segments, where each of the strut segments of the second plurality of strut segments bifurcate to form two strut segments of the third plurality of strut segments. After laser cutting the tubular member to define the support frame, the main portion is expanded to define an expanded configuration of the support frame. Each of the strut segments within the first plurality of strut segments are brought together to form a split collar at an end of the support frame and the split collar is joined together to form the end of the occlusive medical device.

Alternatively or additionally, laser cutting a tubular member may include defining a terminal portion in which each strut segment of the first plurality of strut segments include an arcuate end portion, and the arcuate end portion of each of the first plurality of strut segments combine to form the split collar.

Alternatively or additionally, expanding the main portion may include heat setting the main portion to define the expanded configuration of the occlusive medical device.

Alternatively or additionally, the method may further include securing a membrane to the support frame.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a side view of an example occlusive medical device;

FIG. 2 is a perspective view of a portion of a laser cut tube used in forming an example occlusive medical device such as that shown in FIG. 1;

FIG. 3 is a perspective view of an end portion of the laser cut tube shown in FIG. 2;

FIG. 4 is a schematic side view of a portion of the laser cut tube shown in FIG. 2, shown in a flattened view to illustrate features of the cutting pattern;

FIG. 5 is a schematic side view of the end portion of the cutting pattern shown in FIG. 4;

FIGS. 6A, 6B and 6C are side views illustrating a process by which an end of the example occlusive medical device of FIG. 1 may be formed; and

FIG. 7 is a perspective view of an end portion of an example occlusive medical device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosed invention are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact and/or are affected by the cardiovascular system are prevalent throughout the world. For example, some forms of arterial venous malformations (AVMs) may “feed” off of normal blood flow through the vascular system. Without being bound by theory, it is believed that it may be possible to treat, at least partially, arterial venous malformations and/or other diseases or conditions by starving them of normal, oxygen and/or nutrient-rich blood flow, thereby limiting their ability to grow and/or spread. Other examples of diseases or conditions that may benefit from vascular occlusion include, but are not limited to, bleeds, aneurysms, venous insufficiency, shutting off blood flow prior to organ resection, or preventing embolic bead reflux into branch vessels in the liver. Disclosed herein are medical devices that may be used within a portion of the cardiovascular system in order to treat and/or repair some arterial venous malformations and/or other diseases or conditions. The devices disclosed herein may also provide a number of additional desirable features and benefits as described in more detail below.

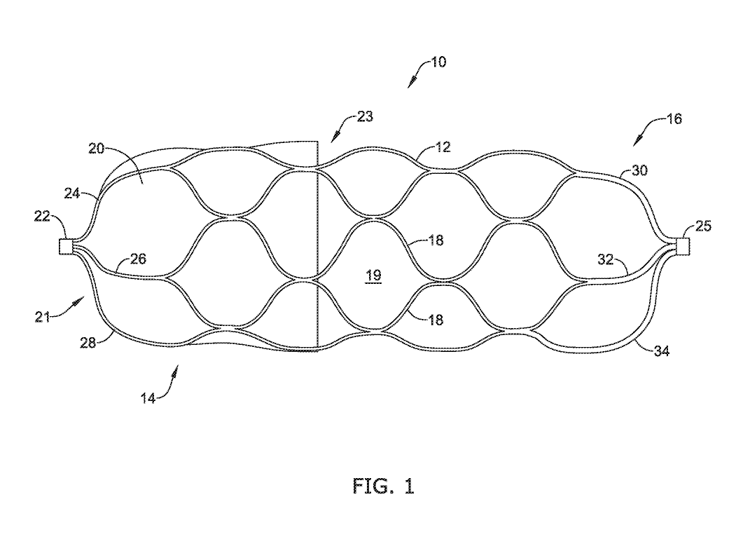

FIG. 1 is a side view of an illustrative but non-limiting occlusive medical device 10 that may, for example, be deployed within a patient's cardiovascular system in order to at least temporarily, if not permanently, stop blood flow through a particular vessel. In some cases, the occlusive medical device 10 may include a frame 12 and a membrane 20 that is fixedly attached to, encapsulating and/or surrounding at least a portion of the frame 12. In some instances, the membrane 20 may be impermeable or at least substantially impermeable to blood flow through the membrane 20. In some cases, the membrane 20 may include one or more vent holes that allow some blood flow therethrough. In some cases, the occlusive medical device 10 may not include the membrane 20.

The frame 12 extends from a proximal region 14 to a distal region 16 and includes a closed proximal end 22 and a closed distal end 25. It will be appreciated that references to proximal and distal are merely illustrative, as in some cases, the frame 12 could be used in any desired orientation. While the membrane 20 is illustrated as being disposed relative to the proximal region 14 of the frame 12, this is not required in all cases. It will be appreciated that the frame 12 may function to support the membrane 20 (in an expanded configuration as shown) so that the membrane 20 can block flow through the particular vessel in which the occlusive medical device 10 is deployed.

While the occlusive medical device 10 is illustrated in an expanded configuration, it will be appreciated that the occlusive medical device 10 may be temporarily compressed into a compressed configuration for delivery and deployment. Once the occlusive medical device 10 has been delivered to an appropriate location, the occlusive medical device 10 may be expanded into the expanded configuration. In some cases, the occlusive medical device 10 may automatically expand into the expanded configuration as soon as any constraining forces are removed. In other cases, the occlusive medical device 10 may be expanded in the expanded configuration via other expansive forces, such as for example a pull wire that could be secured to the closed distal end 25 and could be pulled to cause the occlusive medical device 10 to expand radially while contracting axially.

In some instances, the membrane 20 may include a generally closed first end 21 that is proximate the closed proximal end 22 of the frame 12 and a generally open second end 23 that is opposite the generally closed first end 21. In some cases, the generally open second end 23 of the membrane 20 may have, as illustrated, a generally straight profile such that the membrane 20 generally has a constant length as measured from the generally closed first end 21 to the generally open second end 23. In some instances, the generally open end 23 of the membrane 20 may have a scalloped or otherwise non-straight profile such that the membrane 20 has a varying length as measured from the generally closed first end 21 to the generally open second end 23. A variety of different shapes and configurations for the membrane 20 are contemplated.

In some cases, the membrane 20 may be substantially non-porous and/or impervious to fluid. For example, in some cases, blood and or other fluids may be unable to pass through the membrane 20. As such, when the occlusive medical device 10 is deployed within a vessel lumen (e.g. an artery, etc.) in an expanded configuration (as shown in FIG. 1), the membrane 20 (as well as the frame 12) may extend across the vessel lumen and substantially and/or completely block and/or occlude fluid blood and/or fluid flow through the vessel lumen. In some cases, the membrane 20 may include or be formed from a knitted, woven and/or porous material where blood quickly coagulates to form an impermeable barrier. Some examples of suitable materials for the membrane 20 include but are not limited to metallic materials, polymeric materials, composite materials, textile materials, and the like.

The frame 12 includes a number of strut segments 18 and may be assembled in any of a variety of ways. In some cases, the frame 12 may be woven or braided together. In some instances, and as will be illustrated, the frame 12 may be cut from a tube and then expanded into the illustrated configuration. In some cases, the frame 12 may be laser cut from a metallic tube. While in some cases the frame 12 may be laser cut from a Nitinol tube, this is not required in all cases. It will be appreciated that in the expanded configuration, the strut segments 18 together define cells 19. In some cases, all of the strut segments 18 may be considered as being about equal in size and shape. In some cases, some of the strut segments 18 may be thinner, thicker, shorter or longer than others of the strut segments 18.

As illustrated, there are a total of three strut segments 24, 26, 28 that join together to form the closed proximal end 22. In some cases, as shown, there are a total of three strut segments 30, 32, 34 that join together to form the closed distal end 25. In other cases, there may be four, five, six or more strut segments that join together to form the closed proximal end 22 and/or to form the closed distal end 25. In some instances, the frame 12 may be considered as being symmetric, with the three strut segments 24, 26, 28 being the same as the three strut segments 30, 32, 34. In some cases, as shown, the three strut segments 30, 32, 34 may be considered as being wider than the three strut segments 24, 26, 28. The process for forming the closed distal end 25 may be the same or different from the process used to form the closed proximal end 22.

In some cases, as will be discussed, the frame 12 may be formed from a laser cut metallic tube that is expanded into an expanded configuration as shown prior to forming the closed distal end 25. In some cases, the cutting pattern created in the metallic tube may include relative size differences in strut segments throughout the frame 12, and particularly at the distal end thereof. In some instances, for example, the strut segments 30, 32, 34 may be thicker, or larger in width, than other strut segments that are located more proximally. As will be discussed, the dimensions of the strut segments 30, 32, 34 relative to the strut segments 18 and/or the strut segments 24, 26, 28 may play a part in how the closed distal end 25 is formed subsequent to an expansion and heat setting process.

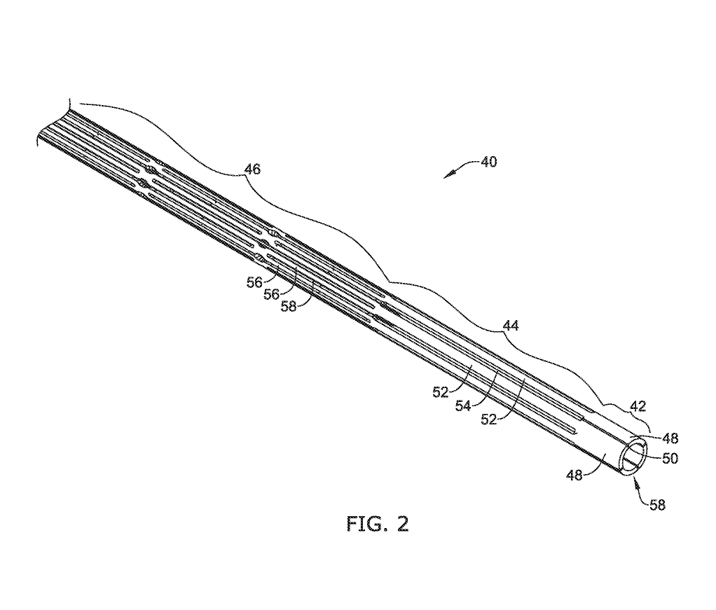

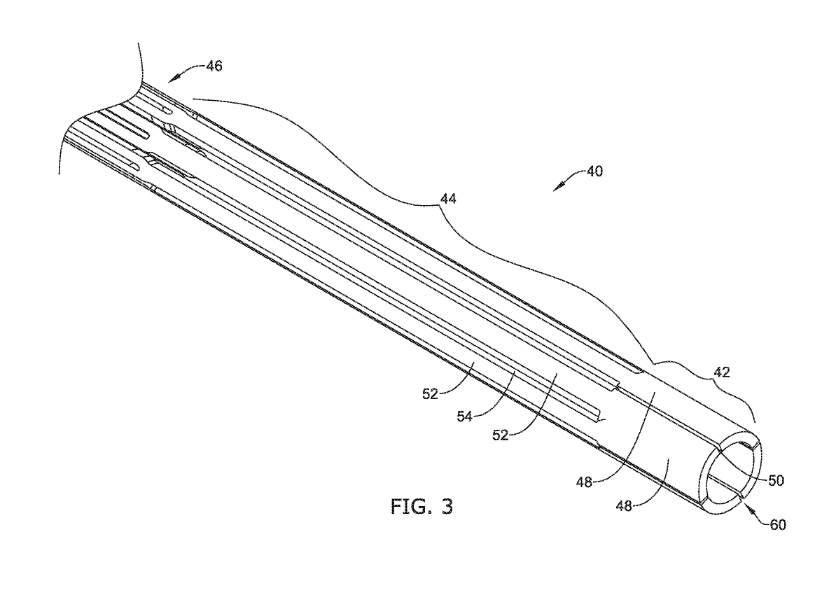

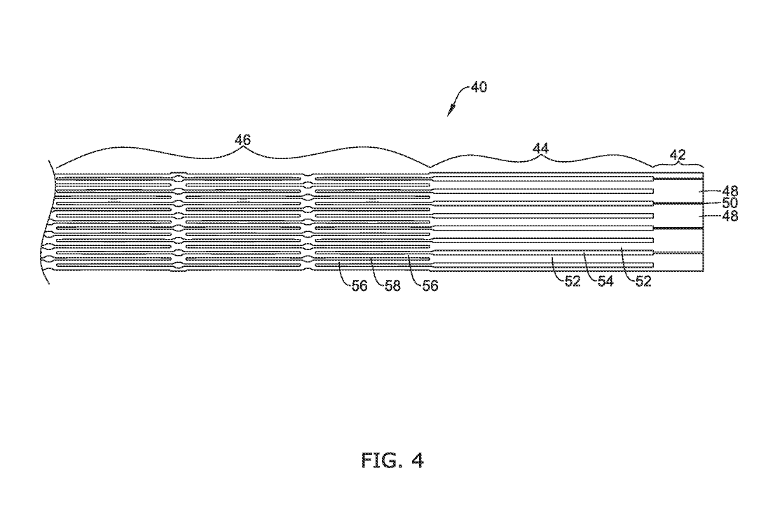

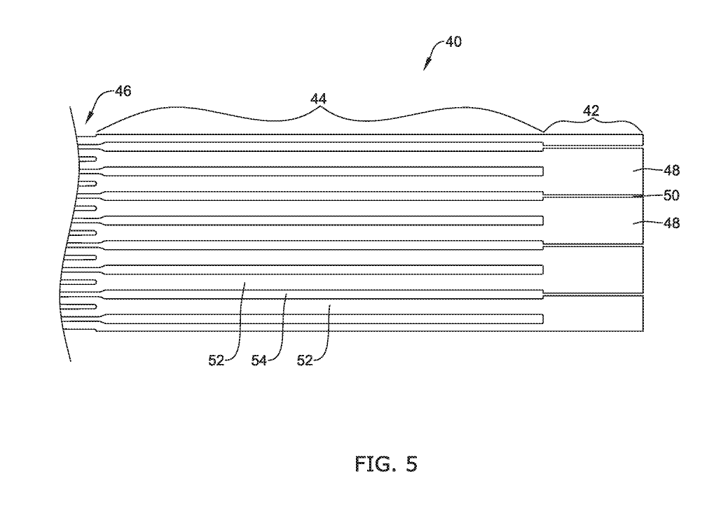

FIG. 2 through FIG. 5 illustrate a laser cutting pattern that may be used in forming the frame 12, including the strut segments 30, 32, 34 being thicker, or larger in width. In this, width may be defined as being a dimension that is measured orthogonally to a length of the particular strut segment. FIG. 2 is a perspective view of a distal portion of a laser cut tube 40 and FIG. 3 is an enlarged perspective view of a portion thereof. FIG. 4 is a corresponding side view, showing the laser cut tube 40 flattened to illustrate the cutting pattern, and FIG. 5 is an enlarged perspective view of a portion thereof. It will be appreciated that FIG. 2 through FIG. 5 essentially provide alternate views of the distal portion of the laser cut tube 40. While FIG. 1 illustrated an occlusive medical device 10 in which a total of three strut segments 30, 32, 34 come together to form the closed distal end 25, in other cases, the closed distal end 25 may be formed from two strut segments coming together, or four, five, six, seven or more strut segments coming together. For example, FIG. 2 through FIG. 5 show a laser cut tube 40 that provides a total of four strut segments that come together to form the closed distal end 25. In some cases, the proximal portion (not illustrated) may be a mirror image of the distal portion. In some instances, the proximal portion may be different, with a different cutting pattern, differently dimensioned struts, etc.

The laser cut tube 40, and thus the resulting occlusive medical device 10 (FIG. 1) may be considered as having a terminal portion 42, an intermediate portion 44 and a main portion 46. In some cases, the terminal portion 42 may be considered as having a first plurality of strut segments 48 that are separated by voids or cuts 50; the intermediate portion 44 may be considered as having a second plurality of struts 52 that are separated by voids or cuts 54; and the main portion 46 may be considered as having a third plurality of struts 56 that are separated by voids or cuts 58. In some cases, the second plurality of strut segments 52 may include a greater number of strut segments than the first plurality of strut segments 48. In some cases, the third plurality of strut segments 56 may include a greater number of strut segments than the second plurality of strut segments 52.

In some cases, as illustrated, each of the strut segments 48 within the terminal portion 42 bifurcates, or becomes two, as one moves from the terminal portion 42 into the intermediate portion 44. In some instances, each of the strut segments 52 within the intermediate portion 44 bifurcates, or becomes two, as one moves from the intermediate portion 44 into the main portion 46. In some cases, and as a result, the intermediate portion 44 may include twice as many strut segments 52 as the number of strut segments 48 within the terminal portion 42. Similarly, the main portion 46 may include, radially about the laser cut tube 40, twice as many strut segments 56 as the number of strut segments 52 within the intermediate portion 44. Alternatively, in some cases, some of the strut segments 48 may not bifurcate, and may instead turn into one or three or more strut segments 52 and/or some of the strut segments 52 may not bifurcate, and may instead turn into one or three or more strut segments 56. These are just examples.

As illustrated, in some cases, each strut segment 52 within the intermediate portion 44 may have a width, measured orthogonally to a length thereof, that is less than half the width of a corresponding strut segment 48 within the terminal portion 42. In some instances, each strut segment 56 within the main portion 46 may have a width, measured orthogonally to a length thereof, that is about half the width of a corresponding strut segment 52 within the intermediate portion 44. Suitable dimensions of the strut segments 48, the strut segments 52 and the strut segments 56 may each be in the range of about 0.0003 inches to about 0.020 inches, or in the range of about 0.001 inches to about 0.010 inches. Moving proximally from the terminal portion 42, the strut segments become progressively smaller while in some cases remaining in the aforementioned size ranges. Put another way, moving distally from the main portion 46, the strut segments become progressively larger. In some cases, for example, this can facilitate formation of the closed distal end 25 (FIG. 1).

In some cases, reducing the number of strut segments in the terminal portion 42, in particular, can make it easier to form the closed distal end 25 because there are fewer strut segments that need to be brought together and secured together to form the closed distal end 25. With particular reference to FIG. 2 and FIG. 3, it can be seen that each of the strut segments 48 form an arcuate end shape. The arcuate end shapes may be thought of as together forming a split collar 60, in which each of the strut segments 48 are separated by voids or cuts 50. In the pre-expanded configuration shown in FIG. 2 and FIG. 3, it can be seen that the strut segments 48 fit together to form the split collar 60. As will be shown in subsequent Figures, the strut segments 48 may be subsequently brought back together after an expansion and heat setting process to once again form the split collar 60.

Once the laser cut tube 40 has been formed, the laser cut tube 40 may be subjected to an expansion and heat setting process which serves to progressively expand the laser cut tube 40. In some cases, the expansion and heat setting process may be a single step or a multiple step process in which progressively larger shaping tools are inserted into an interior of the laser cut tube 40. Once the laser cut tube 40 has been fully expanded, or expanded to a desired expanded configuration, the laser cut tube 40 may be heat set in order to lock in the expanded configuration, especially if the laser cut tube 40 is formed from a shape memory material such as Nitinol. In some cases, the closed proximal end 22 (FIG. 1) may be formed prior to the expansion process, using any desired process or technique. In some instances, the closed proximal end 22 may be formed after the expansion process. In some cases, the closed proximal end 22 may be formed using the same process as that used to form the closed distal end 25.

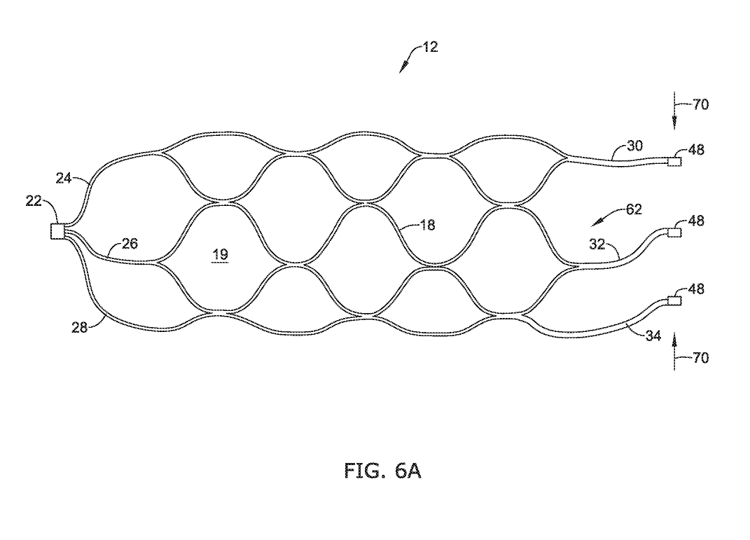

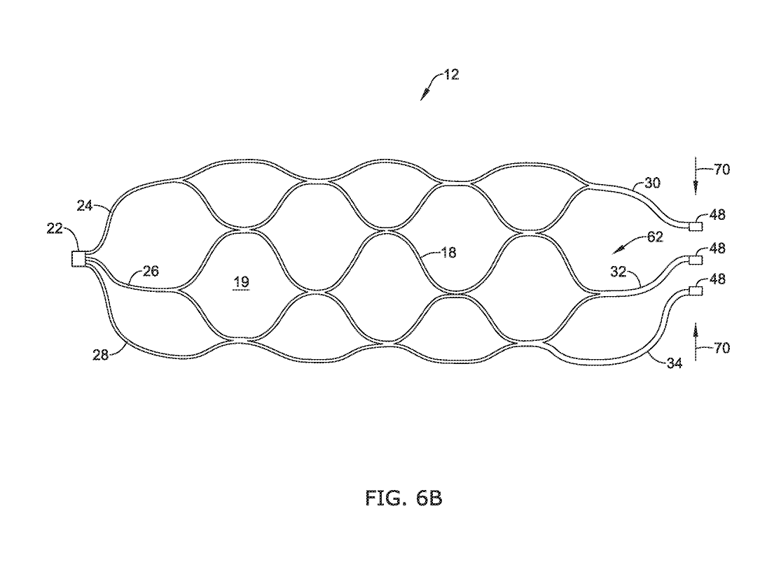

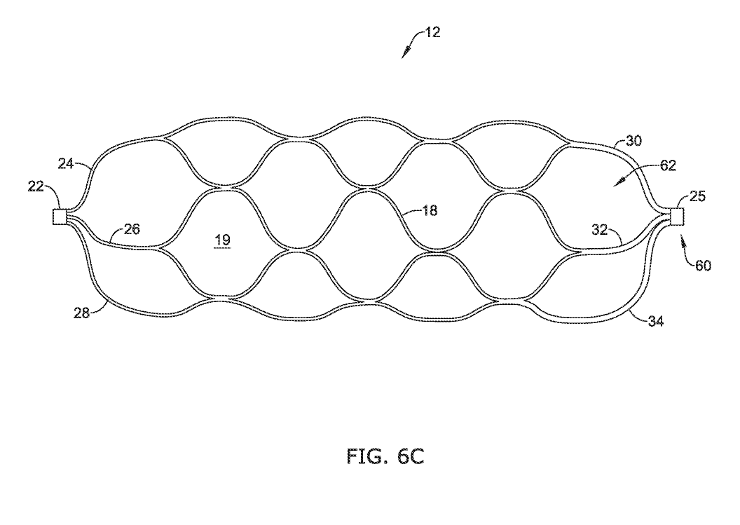

FIG. 6A through FIG. 6C provide an illustrative but non-limiting example of a process for forming the closed distal end 25. In some cases, this process may be used to form the closed proximal end 22, but this is not required in all cases. In FIG. 6A, it can be seen that the strut segments 30, 32, 34, which may be considered as corresponding to the strut segments 52 in the intermediate portion 44 (FIG. 2 through FIG. 5), are still in a relatively open position that would have enabled shaping tools and/or heat setting tools to be loaded into an interior 62. The laser cut tube 40, as shown in FIG. 2, may be expanded into an expanded configuration by inserting shaping tools and/or heat setting tools into the interior 62. By using a sequence of sequentially larger shaping tools and/or heat setting tools, the laser cut tube 40 may be expanded into an expanded configuration as shown in FIGS. 6A through 6C.

The strut segments 52 include a distal end, such as the strut segments 48, that will fit together to form the closed distal end 25. In order to form the closed distal end 25, the strut segments 30, 32, 34 may be subjected to a process in which the strut segments 30, 32, 34 are moved closer together. This is generically represented in FIG. 6A and FIG. 6B as a pair of arrows 70. Moving to FIG. 6B, it can be seen that the strut segments 30, 32, 34 are closer together. In FIG. 6C, it can be seen that the strut segments 30, 32, 34 are closer together, such that the strut segments 48 are joined together to form the split collar 60. Once the split collar 60 is secured together, such as by welding, soldering or the use of adhesive, the closed distal end 25 may be considered as having been formed.

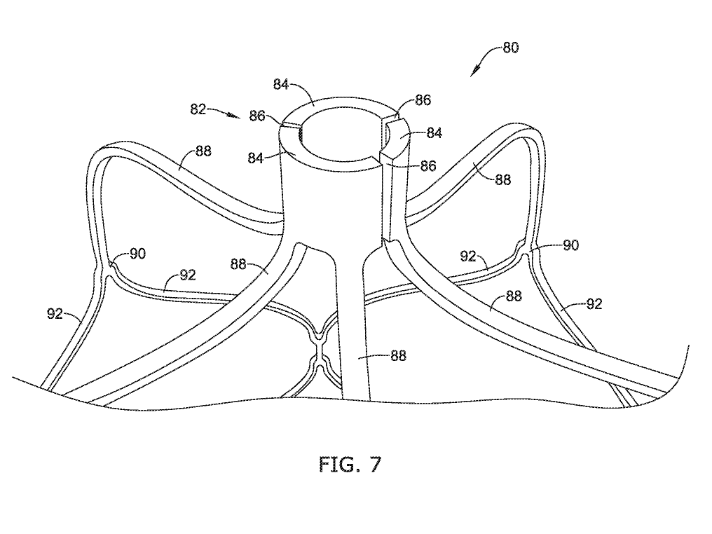

FIG. 7 is an enlarged perspective view of a distal end of a frame 80. The frame 80 may be considered as being representative of the frame 12 shown in FIG. 1 and/or what is represented by the cutting pattern shown in the laser cut tube 40. The frame 80 includes a split collar 82 formed by moving together terminal strut segments 84, each of which are separated by cuts or voids 86. Moving proximally (downward in the illustrated orientation), each of the terminal strut segments 84 may be seen as bifurcating into a pair of intermediate strut segments 88. In some cases, as shown, one of the terminal strut segments 84 may be smaller than the other ones of the terminal strut segments 84, and thus there may only be a single intermediate strut segment 88 extending proximally therefrom. Each of the intermediate strut segments 88 may bifurcate at a bifurcation point 90 into a pair of main strut segments 92.

The occlusive medical device 10, including the frame 12 and/or the occlusive membrane 20, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the outer sheath 12 and the inner catheter 14 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system 10 to achieve the same result.

In some embodiments, a degree of Magnetic Resonance Imaging (MM) compatibility is imparted. For example, the occlusive medical device 10, or portions thereof, may be made of a material that does not substantially distort the image and create substantial artifacts (i.e., gaps in the image). Certain ferromagnetic materials, for example, may not be suitable because they may create artifacts in an MM image. The occlusive medical device 10, or portions thereof, may also be made from a material that the MM machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of the occlusive medical device 10 to aid in delivery and/or to hold the occlusive medical device 10 in a collapsed configuration for delivery until such time as the sheath or covering is removed to allow the occlusive medical device 10 to self-expand into its expanded configuration. The sheath or covering, if present, may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, portions of the occlusive medical device 10 may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, the sheath, if present, may comprise a lubricious, hydrophilic, protective, or other type of coating. Hydrophobic coatings such as fluoropolymers provide a dry lubricity which improves device handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. An occlusive medical device, comprising:
a terminal portion including a first plurality of strut segments that are each joined together at an end thereof to form a closed end of the frame;
an intermediate portion including a second plurality of strut segments, the second plurality of strut segments including a greater number of strut segments than the first plurality of strut segments;
a main portion including a third plurality of strut segments, the third plurality of strut segments including a greater number of strut segments than the second plurality of strut segments;
the occlusive medical device having an expanded configuration in which the second plurality of strut segments and the third plurality of strut segments together form a plurality of cells.

2. The occlusive medical device of claim 1, wherein each strut segment of the first plurality of strut segments in the terminal portion bifurcates into a pair of strut segments of the second plurality of strut segments in the intermediate portion.

3. The occlusive medical device of claim 1, wherein each strut segment of the second plurality of strut segments in the intermediate portion bifurcates into a pair of strut segments of the third plurality of strut segments in the main portion.

4. The occlusive medical device of claim 1, wherein each of the first plurality of strut segments are configured to come together to form part of a split collar.

5. The occlusive medical device of claim 4, wherein each of the first plurality of strut segments include an arcuate end portion, and the arcuate end portion of each of the first plurality of strut segments combine to form the split collar.

6. The occlusive medical device of claim 5, wherein the arcuate end portion of each of the first plurality of strut segments are joined together to form an end of the occlusive medical device.

7. The occlusive medical device of claim 1, further comprises a closed proximal end proximal of the main portion of the occlusive medical device.

8. The occlusive medical device of claim 1, further comprising a membrane.

9. The occlusive medical device of claim 1, wherein the occlusive medical device defaults to the expanded configuration when unconstrained.

10. An occlusive medical device, comprising:
a membrane; and
a support frame supporting the membrane, the support frame including:
a terminal portion including a first plurality of strut segments that are each joined together at an end thereof to form a closed end of the frame, each of the first plurality of strut segments having a first width, the first width measured orthogonally to a length of each of the first strut segments;
an intermediate portion including a second plurality of strut segments, each of the second plurality of strut segments having a second width that is less than the first width, the second width measured orthogonally to a length of each of the second strut segments;
a main portion including a third plurality of strut segments, each of the third plurality of strut segments having a third width that is less than the second width, the third width measured orthogonally to a length of each of the third strut segments.

11. The occlusive medical device of claim 10, wherein the second width is less than half of the first width.

12. The occlusive medical device of claim 10, wherein the third width is about half of the second width.

13. The occlusive medical device of claim 10, wherein each strut segment of the first plurality of strut segments in the terminal portion bifurcates into a pair of strut segments of the second plurality of strut segments in the intermediate portion.

14. The occlusive medical device of claim 10, wherein each strut segment of the second plurality of strut segments in the intermediate portion bifurcates into a pair of strut segments of the third plurality of strut segments in the main portion.

15. The occlusive medical device of claim 10, wherein each of the first plurality of strut segments are configured to come together to form part of a split collar.

16. The occlusive medical device of claim 15, wherein each of the first plurality of strut segments form an arcuate end portion, and the arcuate end portion of each of the first plurality of strut segments combine to form the split collar.

17. A method of manufacturing an occlusive medical device, the method comprising:
laser cutting a tubular member to define a support frame including:
a terminal portion including a first plurality of strut segments;
an intermediate portion including a second plurality of strut segments, where each of the strut segments of the first plurality of strut segments bifurcate to form two strut segments of the second plurality of strut segments;
a main portion including a third plurality of strut segments, where each of the strut segments of the second plurality of strut segments bifurcate to form two strut segments of the third plurality of strut segments;
expanding the main portion to define an expanded configuration of the support frame;
bringing together each of the strut segments within the first plurality of strut segments to form a split collar at an end of the support frame; and
joining together the split collar to form the end of the occlusive medical device.

18. The method of claim 17, wherein laser cutting a tubular member comprises defining a terminal portion in which each strut segment of the first plurality of strut segments include an arcuate end portion, and the arcuate end portion of each of the first plurality of strut segments combine to form the split collar.

19. The method of claim 17, wherein expanding the main portion comprises heat setting the main portion to define the expanded configuration of the occlusive medical device.

20. The method of claim 17, further comprising securing a membrane to the support frame.
